(19) United States
(12) Patent Application Publication
(10) Pub. No.: US 2026/0251542 A1
(43) Pub. Date: Aug. 27, 2026

(54) URINE, SALIVA, AND/OR MOUTHWASH SAMPLE PREPARATION SYSTEM

(71) Applicant: Testmate Health SA, Vaud (CH)

(72) Inventors: Louis Neveu, Yverdon-les-bains (CH); Gaëtan Cherbuin, Chavannes-près-Renens (CH); Hüseyin Baris Atakan, Rotkreuz (CH); Noa Schmid, Alpnach (CH); David Schmid, Untervaz (CH); Elie Graham, Rolle (CH)

(21) Appl. No.: 18/877,985

(22) PCT Filed: Jun. 20, 2023
(86) PCT No.: PCT/EP2023/066646

§ 371 Date: Dec. 20, 2024

(30) Foreign Application Priority Data
Jun. 20, 2022 (EP) 22179963.8
Jun. 20, 2022 (EP) 22179966.1

(51) Int. Cl. G01N 1/40

(52) U.S. Cl. CPC G01N 1/4077; G01N 2001/4088

(57) ABSTRACT

The present invention relates to a system and method for processing a urine, saliva or mouthwash sample for testing for one or more sexually transmitted diseases, oncological conditions, fungal infections, and/or bacterial infections. The system comprises a barrel having an open end and a chamber for holding the sample and fluid conduits; an outlet; a filter positioned between the chamber and the outlet having a pore size for allowing fluid flow and for obstructing cells; an inlet; and reagents selected from a washing buffer, a lysis buffer, a neutralization buffer, and a rehydration buffer. Each of the reagents is provided sequentially through the fluid conduits to the filter. The sample preparation system has a reception configuration in which the sample can be introduced into the barrel and a preparation configuration in which the chamber is at least partially compressed, thereby forcing the sample through the filter.

### BACKGROUND

The invention relates to a sample preparation system for processing a urine, saliva and/or mouthwash sample for testing for one or more sexually transmitted diseases, oncological conditions and/or other pathogens in the urine, saliva and/or mouthwash.

Sexually transmitted infections (STIs) represent several of the most frequently reported diseases in Europe and in the United States and, notably, the incidence of chlamydia and gonorrhea have increased in recent years. Notably, during the Covid-19 pandemic access to clinics and doctors was sharpy curtailed. Furthermore, many individuals were hesitant to have in-person appointments with healthcare providers given the risk of Covid-19 transmission. As such, experts believe that many sexually transmitted infections went unidentified during this time. Many patients with a sexually transmitted infection can be asymptomatic and further transmit the infection before the infection is diagnosed and treated. For some the stigma associated with STIs can present barriers to patients seeking treatment. Even those who seek testing and treatment may have difficulty with access to testing and delays in receiving test results. Altogether these factors provide numerous obstacles to diagnosis and treatment which put individuals at risk for acquiring a sexually transmitted infection or having further complications from unidentified infections.

The testing market is changing to address some of these barriers by increasing the speed of testing, such a same-day testing being available in doctor's offices and clinics. Additionally, at home sampling kits allow people to order a kit and self-sample in a private location and then mail the sample to a central lab without needing to visit a doctor or clinic. However, these home sampling kits generally take days or weeks to receive the results. Thus, a need exists which can provide testing in the privacy of a person's home and fast at-home results.

However, one of the main limiting factors for same-day home testing is the ability to prepare samples for testing. Unlike pregnancy tests, for example, which are able to sample the concentration of free-floating hormones present within urine or other samples, in order to test for sexually transmitted infection, the DNA content present within e.g. bacterial cells or protists must be made accessible and must have a high enough concentration to be detectable. These technical challenges are addressed with the following inventive system and methods.

Consequently, it is an object of the present invention to provide a system which provides an improvement over known processes and enables for sample preparation for use in DNA or RNA amplification from urine, saliva and/or mouthwash samples.

### SUMMARY

This object is achieved with the features of the independent claims. Dependent claims refer to preferred embodiments.

According to a first aspect, the invention relates to a sample preparation system for processing a urine sample, a saliva sample and/or a mouthwash sample. Preferably, the sample preparation system serves for processing the urine, saliva and/or mouthwash sample for testing for one or more sexually transmitted diseases (such as infections caused by bacteria—e.g. chlamydia or gonorrhea—or infections caused by protists—such as e.g. trichomonas vaginalis), oncological conditions (e.g., cancerous or precancerous conditions, such as for example conditions to be determined based on the presence of cancerous or precancerous cells in the urine, saliva or mouthwash), bacterial infections (such as urinary tract infection, urethritis, cystitis, pyelonephritis, gingivitis, and/or periodontitis), fungal infections, and/or protist infections.

The sample preparation system comprises a barrel having an open end and further comprising a chamber for holding the sample and one or more fluid conduits, an outlet, a filter positioned between the chamber and the outlet, the filter having a pore size suitable for allowing fluid flow therethrough and for obstructing the flow of cells (e.g., bacterial, protist, or bodily cells) or fungi. The system (e.g., the barrel) comprises an inlet for receiving the sample into the chamber, and one or more reagents selected from the group comprising a washing buffer, a lysis buffer, a neutralization buffer, and a rehydration buffer, wherein the system is configured to be actuated such that each of the one or more reagents is provided sequentially through the one or more fluid conduits to the filter. The system may also be considered to form a sample preparation unit or kit in the context of the present invention.

Preferably, the sample preparation system has a reception configuration and a preparation configuration, the system being movable between the reception configuration in which the sample can be introduced into the barrel, and the preparation configuration in which the chamber is at least partially compressed, thereby forcing the sample through the filter.

Preferably, the system is configured to force the sample out of the outlet when moved from the reception configuration to the preparation configuration. It is further preferred that the system comprises a piston configured to force the sample through the outlet. Discharging the unused portion of the sample allows for the remaining cellular components to be concentrated on the filter. A piston can help to guide this process more efficiently and prevent any backflow.

Preferably, the system further comprises a cap configured for mounting on the open end of the barrel. The cap may comprise the inlet for receiving the sample into the chamber. In some configurations the piston is supported in the cap. The cap may be mounted on the barrel via a threaded connection. The cap may be partially screwed on to the barrel in the reception configuration. The cap may be further and/or fully screwed onto the barrel in the preparation configuration. Screwing the cap onto the barrel may move the piston through the chamber. A cap which is screwable onto the barrel enables an easy transition between the reception configuration and the preparation configuration which can be performed by a home user. It will be appreciated that instead of a threaded connection any other mechanism allowing a relative movement (in particular, a translatory movement) between the barrel and the cap may be used, e.g. a bayonet connections or a sliding arrangement wherein a projection of the cap extends into a groove of the barrel (or vice-versa).

The barrel and/or cap may comprise a locking arrangement that hinders or prevents a reverse movement of the piston and/or cap once it has been moved from the reception configuration to the preparation configuration. This may prevent the user from manipulating the system incorrectly.

Preferably, the barrel comprises an outlet conduit connecting the chamber to the outlet, wherein the one or more fluid conduits are configured to deliver the reagents into the outlet conduit. Alternatively, the one or more fluid conduits may be configured to deliver the reagents directly onto the filter.

Preferably each of the reagent containers is in fluid communication, or configured to be brought into fluid communication, with a separate fluid conduit of the one or more fluid conduits.

Preferably, each of the reagent containers is configured to be actuated separately by a separate actuator. Separate application of the reagents allows for cleaning and preparation of the cellular contents for analysis.

Preferably, the filter has a pore size no greater than 1.0 μm, more preferably no greater than 0.8 μm, even more preferably no greater than 0.6 μm, most preferably no greater than 0.5 μm. Preferably the filter has a pore size no greater than 0.45 μm, more preferably no greater than 0.4 μm, and most preferably no greater than 0.20 μm. This pore size is suitable for retaining bacterial cells or other analytes of interest (e.g., protists, fungi, cancerous or precancerous cells) and transmitting other unwanted components of the sample.

Preferably, the outlet comprises at least one further filter. The further filter may also be referred to as a pre-filter herein.

The pre-filter preferably has a pore size may be no greater than 250 μm, 150 μm, or 80 μm. Alternatively, the pore sizes of the pre-filter 250 may be no less than 0.6 μm, 0.8 μm, 1.0 μm, 10 μm, or 20 μm. The pre-filter may be place prior to the main filter such that larger particles or conglomerations may be strained out before arriving at, and potentially blocking the main filter. The pre-filter may be arranged in the flow direction of the sample before the chamber.

In some embodiments, the sample preparation system further comprises an outlet cap configured to couple with the barrel, preferably wherein the outlet cap is configured to hold the filter in place, and/or preferably wherein the outlet is formed in the outlet cap.

Preferably the system is configured to receive between 1 mL and 100 mL of sample, preferably between 5 mL and 50 mL of sample, more preferably between 8 and 12 mL of sample, most preferably around 10 mL of sample. A large sample allows for a higher concentration of any possible bacteria (or other analyte of interest) after filtering of the sample. Further, providing a larger sample may be more convenient for the user.

Preferably, the one or more reagents comprise the washing buffer. The washing buffer may comprise purified water. The washing buffer assists in removing unwanted components of the sample. In some configurations delivery of the washing buffer to the filter may be initiated simultaneously with the actuation of the sample preparation device from the reception configuration to the preparation configuration.

Preferably the one or more reagents comprise the lysis buffer. The lysis buffer may comprise at least one of sodium hydroxide, sodium dihydrogen phosphate, disodium hydrogen phosphate, Tris, HEPES, and detergent, more preferably wherein the lysis buffer comprises at least 250 mM NaOH. The lysis buffer may disrupt the cellular membrane and allows the genetic material of any potential bacterial cells or other analyte of interest to be accessed.

Preferably one or more reagents comprise the neutralization buffer. The neutralization buffer may comprise a composition for neutralization of the pH value of the sample, preferably wherein the neutralization buffer comprises one of Tris, Tris-HCl, and ethylenediaminetetraacetic acid (EDTA). In particular the neutralization buffer may serve to lower and/or neutralize the pH value of the lysate obtained after adding the lysis buffer.

Preferably the one or more reagents comprise a rehydration buffer. The rehydration buffer may comprise purified water, more preferably wherein the rehydration buffer comprises one or more of KCl, ammonium sulfate, MgSO4, deoxynucleotide triphosphates, detergent, Triton X-100, and betaine. The rehydration buffer can be used to rehydrate lyophilized pellets containing enzymes and/or oligonucleotides as used in DNA amplification.

According to a further aspect, the invention is also directed to a method of preparing a urine, saliva or mouthwash sample to test for one or more pathogens, preferably one or more sexually transmitted diseases, the method comprising the steps of:
i) collecting the urine sample in a chamber of a sample preparation system;
ii) moving the sample preparation system from a reception configuration into a preparation configuration, thereby flowing the sample through a filter;
iii) actuating the sample preparation system so that one or more reagents are provided through one or more fluid conduits to the filter.

Preferably, the method employs the sample preparation system according to the first aspect of the invention described above.

Preferably, actuating the system comprises actuating at least one actuator of the system, preferably actuating a plurality of actuators of the system.

Preferably, actuating the system comprises providing a plurality of the reagents sequentially to the filter.

In some configurations the method may further include the step of iv) fluidically coupling the sample preparation system to an analysis unit configured to test the prepared sample for one or more sexually transmitted diseases.

In some configurations the method step ii) further comprises exerting a pressure on the sample in the chamber. For example, the size of the chamber may be reduced. Preferably, the size of the chamber is controlled by a position of a piston.

Step ii) may comprise moving (e.g., twisting) a cap relative to the barrel. For example, a threaded engagement between the cap and the barrel may reduce the size of the chamber. The piston may be connected to the cap.

Preferably, step iii) comprises sub-steps of
a) actuating the system such that a washing buffer is provided to the filter, preferably by actuating a first actuator of the washing buffer, more preferably by moving the sample preparation system from the reception configuration into the preparation configuration;
b) actuating the system such that a lysis buffer is provided to the filter, preferably by actuating a second actuator connected to the lysis buffer;
c) preferably actuating the system such that a neutralization buffer is provided to the filter, preferably by actuating a third actuator connected to the neutralization buffer; and
d) preferably actuating the system such that a rehydration buffer is provided to the filter, preferably by actuating a fourth actuator connected to the rehydration buffer.

### BRIEF DESCRIPTION OF THE DRAWINGS

The subject matter of the invention will be explained in more detail in the following text with reference to preferred exemplary and non-limiting embodiments which are illustrated in the attached schematic drawings. These figures disclose embodiments of the invention for illustrational purposes only. In particular, the disclosure provided by the figures and description is not meant to limit the scope of protection conferred by the invention.

FIG. 1a depicts a perspective drawing of the sample preparation system;

FIG. 1b is a view of the sample preparation system shown from above;

FIG. 1c is a side view of the sample preparation system;

FIG. 1d is a cross-sectional view of the sample preparation system;

FIG. 2a schematically depicts a perspective view of the barrel portion of the sample preparation system;

FIG. 2b illustrates a top view of the barrel;

FIG. 2c is a bottom view of the barrel;

FIG. 2d is a cross-section of the barrel;

FIG. 2e is a side view of the barrel;

FIG. 3a provides a perspective view of the cap;

FIG. 3b shows a side view of the cap;

FIG. 3c shows a cross-section of the cap;

FIG. 3d provides a top view of the cap;

FIG. 3e provides a bottom view of the cap;

FIG. 4a shows a perspective view of the outlet cap;

FIG. 4b is a side perspective of the outlet cap;

FIG. 4c is a top view of the outlet cap;

FIG. 4d is a bottom view of the outlet cap; and

FIG. 4e is a cross-section of the outlet cap.

### DETAILED DESCRIPTION

The present invention relates to a sample preparation system 100 which is configured to process a sample (e.g., a urine, saliva and/or mouthwash sample) to enable home testing for sexually transmitted infections. As described above, known home STI kits involve a sampling device where one can provide a swab or urine sample. This sampling device is then sent off to a lab for processing. In the present invention, however, the sampling and the processing can be provided in the privacy of one's home. A large barrier to home testing is that the sample must be properly processed before it can be tested. Urine particulates must be filtered out, bacterial matter must be isolated from the urine and then processed to prepare the cellular material for testing. While this process is generally performed in a lab, in the present invention this sample preparation process can be performed simply and privately by a home user. The sample prepared by the sample preparation system 100 described herein can then be provided to a corresponding analysis unit or amplification cassette for readout of results. Amplification can be performed using PCR amplification or using isothermal DNA or RNA amplification methods, e.g. in Loop-Mediated Isothermal Amplification (LAMP). Without wanting to be bound by theory, isothermal amplification is believed to be particularly useful for processing crude lysate.

In the following description the terms “sexually transmitted disease” and “sexually transmitted infection” are used interchangeably to mean an illness which is transmitted through sexual contact between individuals. While the present invention is described in connection with sexually transmitted infections, a person skilled in the art would understand that the present invention may also be used to test for other types of illnesses including, but not limited to urinary tract infection, urethritis, cystitis, pyelonephritis, etc.

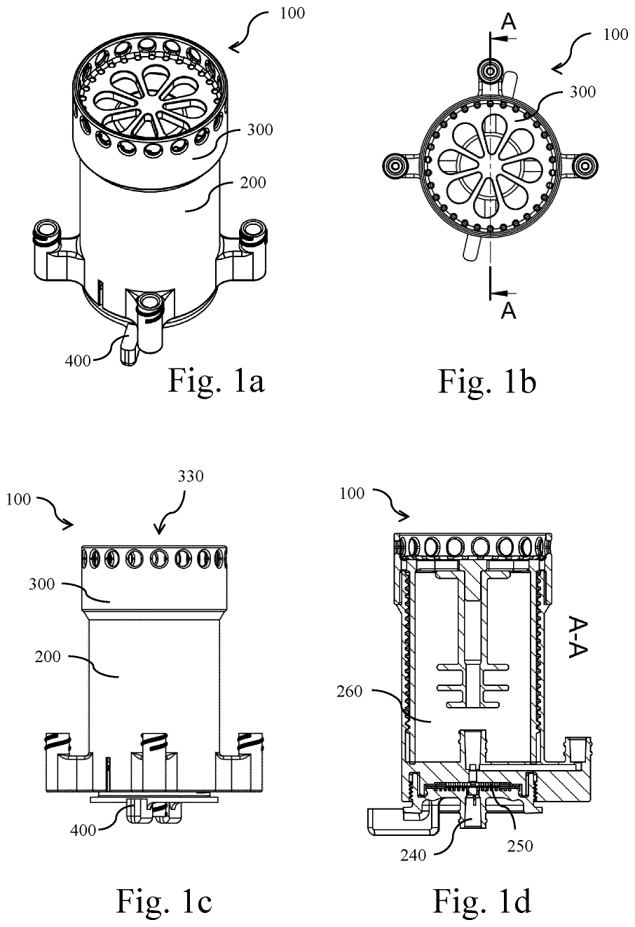

FIG. 1a shows a perspective view of one embodiment of the sample preparation system 100 which includes a cap 300 coupled to a barrel 200. The sample preparation system 100 comprises an inlet 330 which receives a sample (e.g., a urine sample). The sample is then directed to and collected within a chamber 260 in the barrel 200 of the sample preparation system 100. Once the sample has been collected the sample preparation system is actuated to force the sample through a filter 250 which is positioned between the chamber 260 and the outlet 240 which collects cellular material from the sample. Forcing the sample through the filter 250 may also force the sample through the outlet 240. After the sample passes through the filter 250 one or more reagents (not depicted) are actuated so as to introduce specific buffers from the one or more reagents to the collected cellular material on the filter 250. The introduction of the buffers prepares the cellular material from the sample such that it may be used in a separate device (not shown) for PCR amplification and thereby detect a variety of sexually transmitted diseases. In the following description each of these components and steps will be described in more detail.

In the embodiment depicted in FIG. 1a the cap 300 and barrel 200 are shown to be cylindrical, however, one or both of the cap 300 and barrel 200 can have other geometries without impeding the functionality of the sample preparation system 100. The sample preparation system 100 is a home use device which is preferably dimensioned to comfortably fit in the hand of a user.

The sample preparation system may be designed to have an upright orientation with the inlet 330 being positioned above the barrel 200. The sample preparation system 100 is optionally configured to receive between 2 mL and 50 mL of sample, more preferably between 8 and 12 mL of sample, most preferably around 10 mL of sample. A minimum volume of 6 ml or 8 mL (e.g., of whole urine) is advantageous in some circumstances in terms of providing highly accurate results. Larger sample volumes allow for a larger number of potential bacterial or other cells or analytes to be collected on the filter 250 which thereby increases the amount of the subsequent DNA or RNA amplification and the accuracy of STI detection. In some implementations diluted urine or other bodily fluid may also be used. Dilution of the provided sample may be advantageous in reducing pressure, for example during the filtering of the sample. The sample may then be diluted to a ratio of anywhere between 1:1 to 1:50, i.e. between undiluted and one part in fifty. The sample may be diluted to a ratio of 1:2, 1:5, 1:10, 1:20 or 1:50. The sample may be diluted with water.

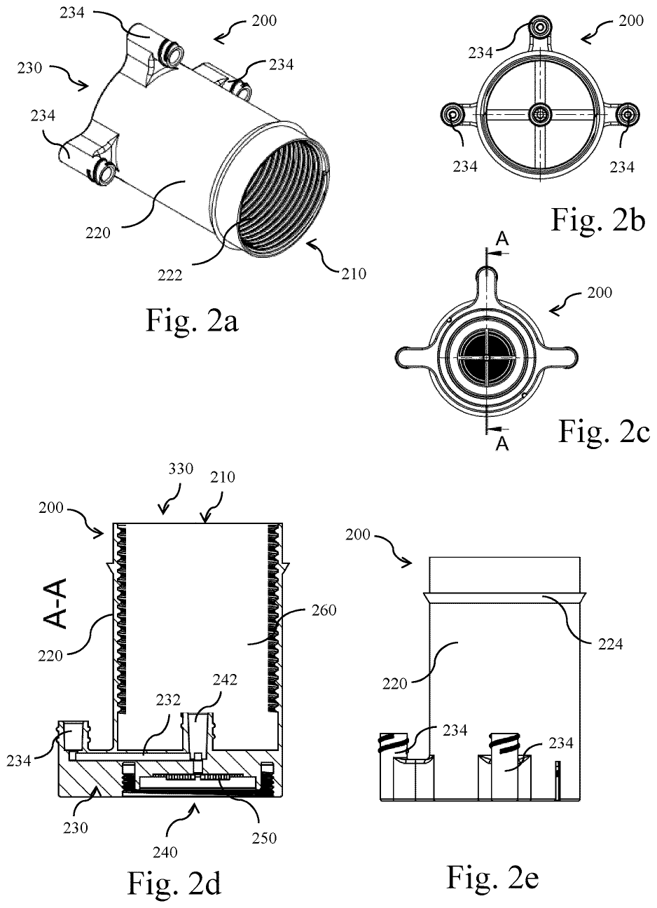

The barrel 200 of one embodiment of the sample preparation system 100 is depicted in FIGS. 2a to 2e having an open side 210 and a base 230. Generally speaking, the barrel 200 may be any shape such that it can accommodate the sample. In preferred configurations of the sample preparation system 100 the barrel 200 has a cylindrical wall 220 extending between the open side 210 and the base 230. Optionally, the open side 210 of the barrel 200 may serve as the inlet 330 for receiving the sample.

The base 230 of the barrel 200 comprises an outlet conduit 242 extending from the chamber 260 to an outlet 240 for the sample. As best viewed in FIG. 2d, a filter 250 is positioned between the chamber 260 and the outlet 240. The filter 250 generally has the smallest pore size sufficient to filter 250 out any cells within the sample, specifically cells of bacterial or protist size which may mean a pore size of maximally 1.0 μm, 0.8 μm, 0.6 μm, 0.5 μm, 0.45 μm, 0.4 μm, or 0.20 μm. The filter 250 may take the form of a membrane or multiple membranes. Where the outlet 240 comprises multiple membranes, including a first membrane closest to the chamber 260 may have a pore size of maximally 150 μm, 100 μm, or 80 μm. A second membrane may have a pore size of maximally 1.0 μm, 0.8 μm, 0.6 μm, 0.5 μm, 0.45 μm, 0.4 μm, or 0.20 μm. And potentially a third membrane furthest from the chamber 260 may be included having a pore size of maximally 150 μm, 100 μm, or 80 μm. Gaskets, particularly silicon gaskets, may be positioned between the membranes and/or above and below the filter 250 in order to hold the filter 250 and the individual membranes in place. The membranes are preferably made out of cellulose acetate (CA), mixed cellulose esters (MCE), polycarbonate track etched (PCTE), and/or cellulose nitrate (CN). Alternatively, the membranes may be made from materials such as mixed cellulose esters (MCE), regenerate cellulose (RC), polytetrafluoroethylene polymer (PTFE), Nylon, cellulose mixed esters (CME), polyvinylidene difluoride (PVDF) and/or polyethersulfone (PES) or similar materials. A membrane having a 0.45 μm pore size and formed from or comprising mixed cellulose esters (MCE) is a preferred embodiment. A membrane having a 0.45 μm pore size and formed from or comprising cellulose acetate (CA) is a preferred embodiment. A membrane having a 0.65 μm pore size or smaller is a preferred embodiment.

When the barrel 200 is used together with a cap 300, the inside of the cylindrical wall 220 may further comprise threads such that it can be screwed together with a compatible cap 300. Further the outside of the cylindrical wall 220 may comprise a stop ridge 224 which limits the advancement of the cap 300.

One particular embodiment of the filter 250 is constructed from a silicon plate of 40 Shore being cut in 20 mm inner diameter, 25 mm outer diameter format at 1 mm and 0.5 mm thickness values to create silicon gaskets 354. 1 mm-thick silicon gasket is glued within the outlet 240 of the barrel 200 by using a silicon glue. An 80 μm pore size nylon net filter 250 is placed over the opening of the barrel 200, just above the 1 mm-thick silicon gasket. A 0.5 mm-thick silicon gasket is placed on top of the nylon net filter 250. A 0.45 μm pore size cellulose acetate (CA) filter 250 is placed on the silicon gasket. Another 0.5 mm-thick silicon gasket is placed on the CA filter 250 and finally a 140 μm pore size nylon net filter 250 is placed.

The presence of a filter 250 positioned between the chamber 260 and the outlet 240 allows for bacterial or other cells of interest to be retained on the filter 250 before passing through the outlet 240. For example, in a healthy individual urine will contain very few or no bacterial cells. However, a person experiencing a sexually transmitted infection may have bacterial growth within the urethra or bladder which is expelled during urination. The pore size of the filter/membranes can thus be optimized to catch bacterial cells protists or fungi, and optionally to further filter out larger material to keep the filter/membrane from becoming obstructed.

The barrel 200 comprises a chamber 260 for receiving the sample. The sample preparation unit comprises a reception configuration and a preparation configuration. In the reception configuration the sample may be introduced into the chamber 260 through the inlet 330 of either the barrel 200 or through a cap 300. After the sample is collected in the chamber 260 the sample preparation system 100 may be transitioned into the preparation configuration. Movement from the reception configuration into the preparation configuration involves the compression of the chamber 260. This may be accomplished in any number of ways, i.e. using valves and/or pumps, such that a pressure is exerted on the liquid within the chamber 260 and that the outlet 240 is provided as the only means of egress for the liquid sample.

In some embodiments the sample preparation system 100 further comprises a receptacle in fluid communication with the outlet 240, so as to receive and store the filtered liquid sample therefrom.

In some embodiments which employ a cap 300, transition between the reception configuration and the preparation configuration involves moving the cap 300 to compress the chamber 260. For example, the cap 300 and the barrel 200 may have corresponding threads which join them, such that in the reception configuration the cap 300 is partially screwed onto the barrel 200 and in the preparation configuration the cap 300 is fully screwed onto the barrel 200. By screwing the cap 300 further onto the barrel 200, the size of the chamber 260 is reduced and the sample is forced through the outlet 240. In some configurations, the inlet 330 is located on the opposite side of the filter 250 from the outlet 240. This configuration provides for more direct throughput such that excess liquid sample which is not retained on the filter 250 can then be directly disposed of, for example, into the toilet.

The pressure exerted on the liquid within the chamber 260 may depend on the speed with which the sample preparation unit is converted from the reception configuration to the preparation configuration by, for example, screwing the cap 300 onto the barrel 200 using threads. The pressure exerted on the liquid within the chamber 260 may also depend on the type of filter used, wherein filters having smaller pore sizes may lead to higher pressures required to convert the sample preparation unit from the reception configuration to the preparation configuration. The pressure exerted on the liquid may be greater than atmospheric pressure. The pressure may be at least 1.5 bar (150 kPa), at least 1.7 bar (170 kPa). The pressure exerted on the liquid is preferably not greater than 6.0 bar (600 kPa), more preferably no greater than 4.0 bar (400 kPa).

If the cap 300 comprises a piston 350, this piston 350 can further aid in forcing the sample through the outlet 240. Moreover, optional gaskets 354 extending radially from the piston 350 can aid in sealing the chamber 260 such that the sample may only flow through the outlet 240. It is noted that throughout the figures the piston 350 is shown without a sealing gasket provided thereon. Such sealing gasket may be connected to the end of the piston which protrudes towards the bottom of the chamber. The gasket may close the gap between the piston and the inner peripheral wall of the chamber 260.

The base 230 of the barrel 200 further may comprise the one or more fluid conduits 232, as can be seen in FIG. 2d. The fluid conduit provides a connection to the reagents which is used for processing the cellular material caught in the filter 250. Each fluid conduit may begin adjacent the filter 250 and lead to a port 234 which serves as the connection point to the one or more reagents. These ports 234 may be adjacent the cylindrical wall 220 of the barrel 200.

The one or more reagents comprise at least a washing buffer, a lysis buffer, a neutralization buffer and a rehydration buffer. The reagents may be provided as a single unit cartridge which may be connected with the single fluid conduit on the barrel 200. Alternatively, the reagents may comprise multiple containers such as syringes, blisters, pouches, etc. which can then individually be attached to multiple fluid conduits 232. In some cases, at least one actuator is provided which initiates the transfer of the buffers from the reagents to the filter 250. In the case of individual syringes, for example, each syringe has a plunger which can be individually actuated to force the buffer through the fluid conduit to the filter 250. Whereas in the case of a reagent blisters or pouches, each blister may be actuated by a user simply compressing the blister(s) and forcing the reagents through the one or more fluid conduits 232 to the filter 250. As such, each respective blister or pouch may be considered to form an actuator. Alternatively, when a single unit cartridge is provided, an actuator button may initiate the process and in conjunction with a microcontroller provide each buffer in a controlled and timed manner.

From the one or more reagents, the washing buffer may comprise purified water. The lysis buffer comprises at least one of sodium hydroxide, sodium dihydrogen phosphate, disodium hydrogen phosphate, Tris, HEPES, and detergent. In a preferred embodiment the lysis buffer comprises at least 250 mM of NaOH. The neutralization buffer is configured to neutralize the pH value of the sample. The neutralization buffer may comprise Tris and/or Tris-HCl. The rehydration buffer comprises one or more of KCl, ammonium sulfate, MgSO₄, deoxynucleotide triphosphates, detergent, Triton X-100, and betaine. Depending on the specific configuration of the sample preparation system 100 any two or more of the washing buffer, lysis buffer, neutralization buffer and rehydration buffer may be combined into a single buffer for delivery to the filter 250. In some embodiments of the present invention a neutralization buffer may be unnecessary or alternatively one reagent mixture may function as a washing buffer, neutralization buffer, and/or rehydration buffer.

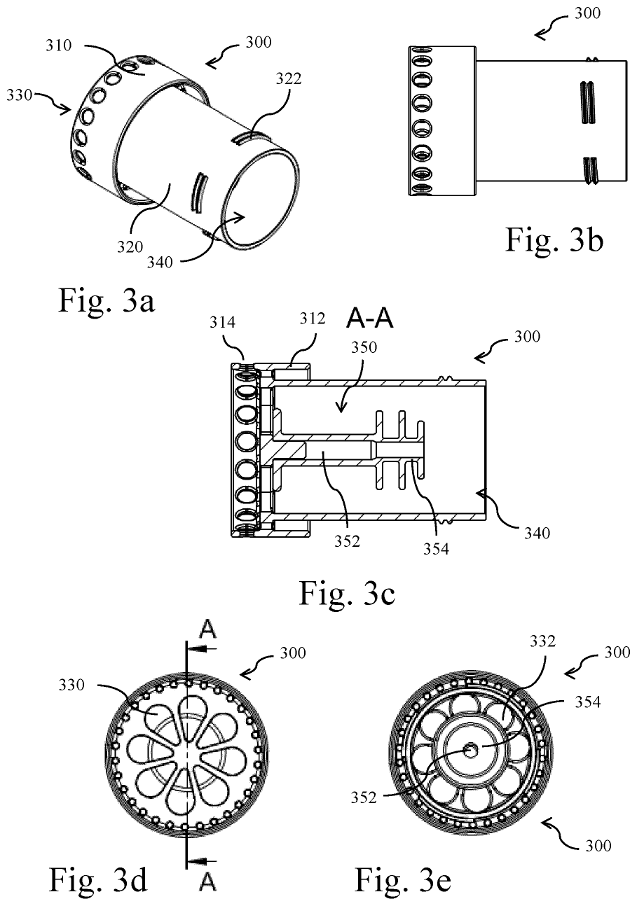

In FIGS. 3a to 3e an embodiment of the cap 300 is schematically depicted. FIG. 3a shows a perspective view of the cap 300 depicted in FIG. 1a. The cap 300 in some embodiments may have a cylindrical form wherein the cylinder comprises a tube 320 with an open end 340 which may be inserted into the barrel 200. The cap 300 may further comprise a top portion 310 which forms a wall or barrier opposite of the open end 340 of the cap 300. In some embodiments the top portion 310 has a larger diameter than that of the tube 320. From the outer circumference of the top portion 310 a lip 312 may extend downwards toward the open end 340 of the cylinder. The larger dimension of the top portion 310 and/or the lip 312 can make it easier for the user to grip the cap 300 and then push or twist it down into the barrel 200.

Further, the cap 300 has an inlet 330 at the top for receiving the sample. The inlet 330 may comprise one or more holes in the cap 300 through which sample can pass. Such configuration may help to guide sample down into the barrel 200.

In the example provided in FIG. 2d the inlet 330 comprises eight petal-shaped holes which are oriented in a radially symmetric pattern around the central axis of the cap 300. It will be appreciated, however, that many other designs are possible in this regard.

Alternatively or additionally, the cap could be provided with an upper funnel (not shown in the figures), which makes it easier to catch the sample (e.g., when whole urine is employed).

Inside the barrel 200 there may be provided a funnel-shaped portion having a wider section and a narrower section. The wider section may serve for guiding the sample into the narrower section. The narrower section may be configured to seal with a piston 350 when the piston 350 is moved through the chamber 260.

From the top portion 310 of the cap 300 a wall may extend from the periphery upwards to create a cup 314 formation. The cup 314 or upper funnel formation makes it easier to collect the sample, prevent splashing and guide the sample into the barrel 200. In some configurations the cup 314 may include a number of holes extending radially outwards through the wall.

In some particularly advantageous embodiments, the cap 300 also comprises a piston 350, as shown in FIG. 3c. The piston 350 can be affixed on the lower face of the top portion 310 and extend within the tube 320 toward the open end 340 of the cap 300. The piston 350 can have an elongated portion 352 which is attached to the top portion 310 of the cap 300 and may further comprise ribs or gaskets 354 extending radially outward therefrom. The gaskets 354 may be rigid or flexible. The piston 350 can assist in forcing the sample out through the outlet 240 of the barrel 200. When a piston 350 is employed the chamber 260 within the barrel 200 may have a smaller diameter than the barrel 200 and corresponding to the diameter of the piston 350 such that the movement of the piston 350 through the chamber 260 blocks the backward flow of the sample. This configuration may also include a funnel positioned within the barrel 200 such that sample is guided from the inlet 330 into the chamber 260 while the sample preparation system 100 is in the reception configuration.

Along the tube 320 one or more threads may extend circumferentially outward. These threads are configured to mate with corresponding threads on the inside of the barrel 200 so that the cap 300 and the barrel 200 can be screwed together.

Additionally, the cap 300 may comprise a pre-filter 332 positioned such that it can filter the sample while it passes from the inlet 330 into the chamber 260. In this way larger particulates can be prevented from entering the chamber 260 for the sample. The pre-filter 332 may also comprise multiple membranes having different pore sizes. In some cases the pore sizes of the pre-filter 332 may be no greater than 250 μm, 150 μm, or 80 μm. Alternatively, the pore sizes of the pre-filter 250 may be no less than 0.6 μm, 0.8 μm, 1.0 μm, 10 μm, or 20 μm. The pore sizes of the pre-filter may be 10 μm, 20 μm, 30 μm, 40 μm, 80 μm, 100 μm, 120 μm, 140 μm, 160 μm, 180 μm, 200 μm or in any range therebetween. In some cases the pre-filter 332 is positioned within the inlet 330 of the cap 300.

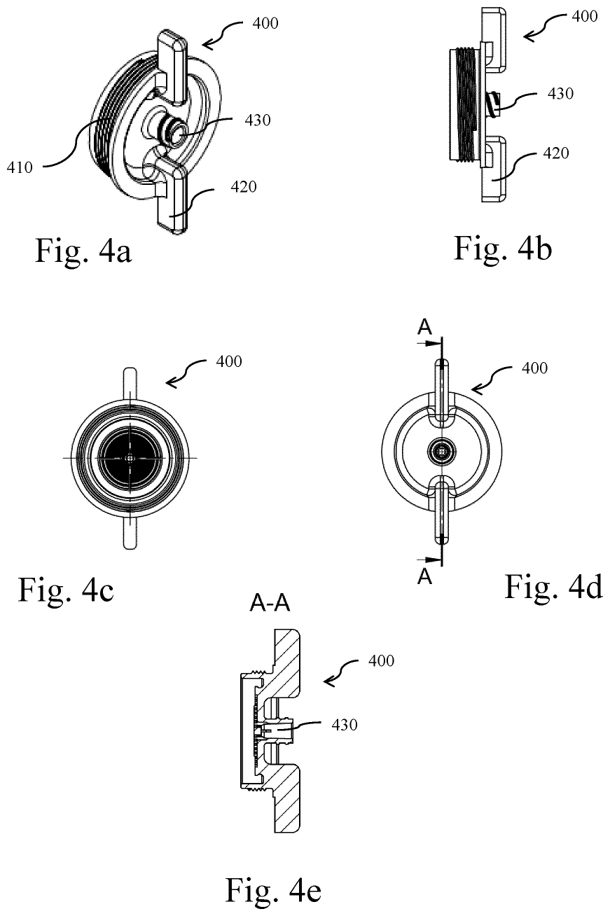

As previously discussed, one or more filters 250 are positioned in the barrel 200, these filters 250 may be held in place by an outlet cap 400 which is depicted in FIGS. 4a to 4e. The outlet cap 400 may have a threaded portion 410 for screwing into a corresponding thread located at the outlet 240 of the barrel 200. Tabs 420 may also be provided on the outlet cap 400 to aid in screwing the outlet cap 400 into the outlet 240. Importantly, the outlet cap 400 comprises a dispensing port 430 which connects to the outlet 240 of the barrel 200, together forming a channel for dispensing the prepared sample. The dispensing port 430 may take the form a Luer-lock or other liquid-tight connection for secure docking of the sample preparation system 100 to an analysis unit or DNA or RNA amplification cassette. Alternatively, the barrel 200 itself may comprise at the outlet 240 or at some other location a docking port for connection to an analysis unit or amplification cassette.

The outlet cap 400 may enable the positioning and replacement of the filter 250 and any gaskets present and may enable easier production of the sample preparation system 100.

A process of preparing the sample to test for sexually transmitted disease or other conditions also forms a part of the present invention. The method comprises the steps of i) collecting the sample in the chamber 260; ii) moving the sample preparation system 100 from the reception configuration into the preparation configuration, thereby flowing the sample through the filter 250; and iii) actuating the at least one actuator so that each of the buffers is provided sequentially through the one or more fluid conduits 232 to the filter 250.

Step i) of the process involves a person who desires to test for a sexually transmitted disease providing a sample into a chamber 260. The sample may be, for example, first catch urine as this likely contains a higher concentration of any bacteria, protist or fungus present. In step ii) the user then transitions the chamber from the reception configuration to the preparation configuration. This transition may involve pushing or screwing the cap 300 onto the barrel 200 which reduces the size of the chamber 260 and forces the sample through the filter 250. This step may be performed over a toilet such that the excess sample may be disposed of leaving any cellular matter trapped on the filter 250. For step iii) the user actuates the one or more reagents which forces the buffers provided therein through the fluid conduits 232 onto the filter 250.

A washing buffer is formulated to rinse away remaining fluid components and/or small molecules (e.g., urobilin) that may act as an amplification inhibitor.

The neutralization buffer is formulated to lower and/or neutralize the pH of the remaining fluid. These buffers allow the cells remaining in the filter 250 to be lysed without suffering negative effects from any remaining sample, e.g. urine (which can contain many bodily waste products and be somewhat acidic). The lysis buffer is provided to the filter 250 to disrupt the cellular membranes of the bacteria (or other analytes of interest), rendering the DNA/RNA contents of the analytes available for binding.

The provision of these buffers to the cellular samples gathered on the filter 250 prepares the sample for introduction into a compatible analysis unit which can test the DNA or RNA sample for a variety of sexually transmitted diseases. It some cases it may be advantageous to connect the sample preparation system 100 to the analysis unit before or during step iii), particularly when a lysis buffer is provided to the cell sample.

In order to promote ease of home use of the sample preparation system 100, instructions for preparing the sample may be provided in a software application running on a mobile telephone or a computer. Such instructions may include the steps of actuation of the sample preparation system 100 including any incubation periods or wait times and the order of actuation of the buffers.

While the invention has been illustrated and described in detail in the drawings and foregoing description, such illustration and description are to be considered illustrative or exemplary and non-restrictive; the invention is thus not limited to the disclosed embodiments. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality and may mean “at least one”.

The following are preferred aspects of the invention:

1. A sample preparation system for processing a urine sample, a saliva sample and/or a mouthwash sample for testing for one or more sexually transmitted diseases, oncological conditions, fungal infections, and/or bacterial infections, the unit comprising:
a barrel having an open end and further comprising a chamber for holding the sample and one or more fluid conduits,
an outlet,
a filter positioned between the chamber and the outlet, the filter having a pore size suitable for allowing fluid flow therethrough and for obstructing the flow of cells (e.g., bacterial cells, body cells, fungal cells and/or protist cells);
an inlet for receiving the sample into the chamber;
one or more reagents selected from the group comprising a washing buffer, a lysis buffer, a neutralization buffer, and a rehydration buffer;
wherein the system is configured to be actuated such that each of the one or more reagents is provided sequentially through the one or more fluid conduits to the filter;
wherein the sample preparation system has a reception configuration and a preparation configuration, the system being movable between the reception configuration in which the sample can be introduced into the barrel, and the preparation configuration in which the chamber is at least partially compressed, thereby forcing the sample through the filter.

2. The sample preparation system of aspect 1, the system being configured to force the sample out of the outlet when moved from the reception configuration to the preparation configuration.

3. The sample preparation system of aspect 1 or 2, wherein the system further comprises a piston configured to force the sample through the outlet.

4. The sample preparation system of aspect 1, 2 or 3, wherein the system further comprises a cap configured for mounting on the open end of the barrel, preferably wherein the cap provides the inlet for receiving the sample into the chamber.

5. the sample preparation system of aspect 4, wherein the piston is supported in the cap.

6. The sample preparation system of aspect 4 or 5, wherein the cap is mounted on the barrel via a threaded connection, the threaded connection having the reception configuration in which the cap is partially screwed on to the barrel and the preparation configuration in which the cap is fully screwed onto the barrel.

7. The sample preparation system of any one of the previous aspects, wherein the one or more fluid conduits are positioned adjacent the filter.

8. The sample preparation system of any one of the previous aspects,
wherein the barrel comprises an outlet conduit connecting the chamber to the outlet, wherein the one or more fluid conduits are configured to deliver the reagents into the outlet conduit; or
wherein the one or more fluid conduits are configured to deliver the reagents directly onto the filter.

9. The sample preparation system of any one of the previous aspects, wherein the inlet further comprises a pre-filter.

10. The sample preparation system of any one of the previous aspects, wherein each of the reagents set is provided in a separate reagent container, for example a syringe or a pouch.

11. The sample preparation system of aspect 10, wherein each of the reagent containers is in fluid communication, or configured to be brought into fluid communication, with a separate fluid conduit of the one or more fluid conduits, preferably wherein each of the reagent containers is configured to be actuated separately, e.g. by a separate actuator.

12. The sample preparation system of any one of the previous aspects, wherein the filter has a pore size no greater than 0.6 μm, preferably no greater than 0.5 μm, more preferably no greater than 0.45 μm, even more preferably no greater than 0.4 μm, and most preferably no greater than 0.2 μm.

13. The sample preparation system of any one of the previous aspects, wherein the outlet comprises at least one further filter, the at least one further filter having a pore size no greater than 150 μm, more preferably no greater than 100 μm, even more preferably no greater than 80 μm.

14. The sample preparation system of aspect 13, wherein each of the filters is separated from the next filter by gaskets and/or held in place by gaskets.

15. The sample preparation system of any one of the previous aspects, wherein the sample preparation system further comprises an outlet cap configured to couple with the barrel.

16. The sample preparation system of aspect 15, wherein the outlet cap is configured to hold the filter in place.

17. The sample preparation system of aspect 15 or 16, wherein the outlet is formed in the outlet cap.

18. The sample preparation system of any one of the previous aspects, wherein the system is configured to receive between 1 mL and 100 mL of sample, preferably between 5 mL and 50 mL of sample, more preferably between 8 and 12 mL of sample, most preferably around 10 mL of sample.

19. The sample preparation system of any one of the previous aspects, wherein the system is configured to receive whole urine, preferably wherein the system is configured to receive whole urine into the chamber or onto the pre-filter.

20. The sample preparation system of any one of the previous aspects, wherein the one or more reagents comprise the washing buffer, preferably wherein the washing buffer comprises purified water.

21. The sample preparation system of any one of the previous aspects, wherein the one or more reagents comprise the lysis buffer, preferably wherein the lysis buffer comprises at least one of sodium hydroxide, sodium dihydrogen phosphate, disodium hydrogen phosphate, Tris, HEPES, and detergent, more preferably wherein the lysis buffer comprises at least 250 mM NaOH.

22. The sample preparation system of any one of the previous aspects, wherein the one or more reagents comprise the neutralization buffer, preferably wherein the neutralization buffer comprises a composition for neutralization of the pH value of the lysate, preferably wherein the neutralization buffer comprises one or more of Tris, Tris-HCl and EDTA.

23. The sample preparation system of any one of the previous aspects, wherein the one or more reagents comprise the rehydration buffer, preferably wherein the rehydration buffer comprises purified water, more preferably wherein the rehydration buffer comprises one or more of KCl, ammonium sulfate, MgSO4, deoxynucleotide triphosphates, detergent, Triton X-100, and betaine.

24. The sample preparation system of any one of the previous aspects, wherein a plurality of the reagents is provided as a reagent set, preferably as a reagent set within a cartridge which may be docked to the one or more fluid conduits.

25. The sample preparation system of any one of the previous aspects, wherein the sample preparation system is configured to be used together with an analysis unit configured to test the prepared sample for a sexually transmitted disease, preferably wherein the sample preparation system is configured to be fluidically connected to an inlet of the analysis unit for transferring the sample.

26. The sample preparation system of the previous aspect, wherein the outlet provides a connector for establishing a fluid connection with the analysis unit, wherein the connector preferably is a liquid-tight connection, more preferably a Luer fitting.

27. A method of preparing a urine sample, a saliva sample and/or a mouthwash sample to test for one or more sexually transmitted diseases, oncological conditions, and/or bacterial infections using the sample preparation system of any of the previous aspects, the method comprising the steps of:
i) collecting the sample in the chamber;
ii) moving the sample preparation system from the reception configuration into the preparation configuration, thereby flowing the sample through the filter;
iii) actuating the system so that the one or more reagents are provided through the one or more fluid conduits to the filter.

28. The method of aspect 27, wherein actuating the system comprises actuating at least one actuator of the system, preferably actuating a plurality of actuators of the system.

29. The method of aspect 27 or 28, wherein actuating the system comprises providing a plurality of the reagents sequentially to the filter.

30. The method of any of aspects 27 to 29, further comprising the step of:
iv) fluidically coupling the sample preparation unit to an analysis unit configured to test the prepared sample for one or more sexually transmitted diseases.

31. The method of any of aspects 27 to 30, wherein the step i) further comprises urinating through the inlet into the chamber of the barrel.

32. The method of any of aspects 27 to 31, wherein the step ii) further comprises twisting a cap relative to the barrel such that a threaded engagement between the cap and the barrel reduces the size of the chamber.

33. The method of any of aspects 27 to 32, wherein the size of the chamber is controlled by the position of a piston connected to the cap.

34. The method of any of the aspects 27 to 33, wherein step iii) comprises sub-steps of
a) actuating the system such that a washing buffer is provided to the filter, preferably by actuating a first actuator of the washing buffer, more preferably by moving the sample preparation system from the reception configuration into the preparation configuration;
b) actuating the system such that a lysis buffer is provided to the filter, preferably by actuating a second actuator connected to the lysis buffer;
c) preferably actuating the system such that a neutralization buffer is provided to the filter, preferably by actuating a third actuator connected to the neutralization buffer; and
d) preferably actuating the system such that a rehydration buffer is provided to the filter, preferably by actuating a fourth actuator connected to the rehydration buffer.

35. The method of any of the aspects 27 to 34, wherein step ii) further comprises prokaryotic and/or eukaryotic cells becoming trapped on the filter.

36. The method of any of the aspects 27 to 35, wherein step i) further comprises introducing a whole and/or fresh urine sample into the chamber or onto a pre-filter in the inlet.

1. A sample preparation system (100) for processing a urine sample, a saliva sample and/or a mouthwash sample for testing for one or more sexually transmitted diseases, oncological conditions, fungal infections, and/or bacterial infections, the unit comprising:
a barrel (200) having an open end and further comprising a chamber (260) for holding the sample and one or more fluid conduits (232);
an outlet (240);
a filter (250) positioned between the chamber (260) and the outlet (240), the filter (250) having a pore size suitable for allowing fluid flow therethrough and for obstructing the flow of cells;
a cap (300) configured for mounting on the open end of the barrel (200);
an inlet (330) at the open end of the barrel (200) for receiving the sample into the chamber (260);
one or more reagent containers comprising one or more reagents selected from the group comprising a washing buffer, a lysis buffer, a neutralization buffer, and a rehydration buffer;
wherein the system (100) is configured to be actuated such that each of the one or more reagents is provided sequentially through the one or more fluid conduits (232) to the filter (250);
wherein the sample preparation system (100) has a reception configuration and a preparation configuration, the system being movable between the reception configuration in which the sample can be introduced into the barrel (200), and the preparation configuration in which the chamber (260) is at least partially compressed, thereby forcing the sample through the filter (250); and
wherein the cap (300) is mounted on the barrel (200) via a threaded connection wherein screwing the cap (300) onto the barrel (200) transitions the system (100) from the reception configuration to the preparation configuration, and wherein transitioning the system (100) from the reception configuration to the preparation configuration also forces the sample through the outlet (240).

2. The sample preparation system of claim 1, the system (100) being configured to force the sample out of the outlet (240) when moved from the reception configuration to the preparation configuration, preferably wherein the system further comprises a piston (350) configured to force the sample through the outlet (240).

3. The sample preparation system of claim 1 or claim 2, preferably wherein the cap (300) provides the inlet (330) for receiving the sample into the chamber; preferably wherein the piston (350) is supported in the cap (300); more preferably wherein the cap (300) is mounted on the barrel (200) via a threaded connection, the threaded connection having the reception configuration in which the cap (300) is partially screwed on to the barrel (200) and the preparation configuration in which the cap (300) is fully screwed onto the barrel (200).

4. The sample preparation system of any one of the previous claims,
wherein the barrel (200) comprises an outlet conduit (242) connecting the chamber (260) to the outlet (240), wherein the one or more fluid conduits (232) are configured to deliver the reagents into the outlet conduit (242); or
wherein the one or more fluid conduits (232) are configured to deliver the reagents directly onto the filter (250); preferably wherein each of the reagent containers is in fluid communication, or configured to be brought into fluid communication, with a separate fluid conduit of the one or more fluid conduits (232), preferably wherein each of the reagent containers is configured to be actuated separately by a separate actuator.

5. The sample preparation system of any one of the previous claims, wherein the filter (250) has a pore size no greater than 1.0 μm, preferably no greater than 0.8 μm, more preferably no greater than 0.6 μm, even more preferably no greater than 0.5 μm, most preferably no greater than 0.45 μm; alternatively the filter (250) has a pore size no greater than 0.4 μm, and preferably no greater than 0.20 μm; and/or wherein the outlet (240) comprises at least one further filter, the at least one further filter having a pore size no greater than 150 μm, more preferably no greater than 100 μm, even more preferably no greater than 80 μm.

6. The sample preparation system of any one of the previous claims, wherein the sample preparation system (100) further comprises an outlet cap (400) configured to couple with the barrel (200) wherein the outlet cap (400) is configured to hold the filter (250) in place, and wherein the outlet is formed in the outlet cap (400).

7. The sample preparation system of any one of the previous claims, wherein the system (100) is configured to receive between 1 mL and 100 mL of sample, preferably between 5 mL and 50 mL of sample, more preferably between 8 and 12 mL of sample, most preferably around 10 mL of sample.

8. The sample preparation system of any one of the previous claims, wherein the one or more reagents comprise the washing buffer, preferably wherein the washing buffer comprises purified water.

9. The sample preparation system of any one of the previous claims, wherein the one or more reagents comprise the washing buffer preferably comprising purified water and/or wherein the one or more reagent comprise the lysis buffer, preferably wherein the lysis buffer comprises at least one of sodium hydroxide, sodium dihydrogen phosphate, disodium hydrogen phosphate, Tris, HEPES, and detergent, more preferably wherein the lysis buffer comprises at least 250 mM NaOH.

10. The sample preparation system of any one of the previous claims, wherein the system (100) is configured such that transitioning the system (100) from the reception configuration to the preparation configuration allows any excess sample to be simultaneously disposed of, preferably disposed of into the toilet.

11. The sample preparation system of any one of the previous claims, wherein the one or more reagents comprise the rehydration buffer, preferably wherein the rehydration buffer comprises purified water, more preferably wherein the rehydration buffer comprises one or more of KCl, ammonium sulfate, MgSO4, deoxynucleotide triphosphates, detergent, Triton X-100, and betaine.

12. A method of preparing a urine, saliva or mouthwash sample to test for one or more sexually transmitted diseases using the sample preparation system (100) of any of the previous claims, the method comprising the steps of:
i) collecting the sample in the chamber (260);
ii) moving the sample preparation system (100) from the reception configuration into the preparation configuration, thereby flowing the sample through the filter (250);
iii) actuating the system (100) so that the one or more reagents are provided through the one or more fluid conduits (232) to the filter (250).

13. The method of claim 12, wherein actuating the system (100) comprises actuating at least one actuator of the system, preferably actuating a plurality of actuators of the system; and/or wherein actuating the system comprises providing a plurality of the reagents sequentially to the filter (250).

14. The method of claim 12 or claim 13, further comprising the step of:
iv) fluidically coupling the sample preparation unit to an analysis unit configured to test the prepared sample for one or more sexually transmitted diseases; and/or wherein the step ii) further comprises twisting a cap (300) relative to the barrel (200) such that a threaded engagement between the cap (300) and the barrel (200) reduces the size of the chamber (260); and/or wherein the size of the chamber (260) is controlled by the position of a piston (350) connected to the cap (300).

15. The method of any of the claims 12 to 14, wherein step iii) comprises sub-steps of
a) actuating the system (100) such that a washing buffer is provided to the filter (250), preferably by actuating a first actuator of the washing buffer, more preferably by moving the sample preparation system (100) from the reception configuration into the preparation configuration;
b) actuating the system (100) such that a lysis buffer is provided to the filter (250), preferably by actuating a second actuator connected to the lysis buffer;
c) preferably actuating the system (100) such that a neutralization buffer is provided to the filter (250), preferably by actuating a third actuator connected to the neutralization buffer; and
d) preferably actuating the system (100) such that a rehydration buffer is provided to the filter (250), preferably by actuating a fourth actuator connected to the rehydration buffer.
